(19) United States
(12) Patent Application Publication
(10) Pub. No.: US 2009/0136440 A1
(43) Pub. Date: May 28, 2009

(54) Mixtures of phosphorus-containing compounds, a process for their preparation, and their use as flame retardants

(75) Inventors: Wiebke Maas, Huerth (DE); Mathias Dietz, Simmerath (DE); Martin Sicken, Koeln (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd.

(21) Appl. No.: 12/313,542

(22) Filed: Nov. 21, 2008

(30) Foreign Application Priority Data
Nov. 23, 2007 (DE) 10 2007 056 532.3

(51) Int. Cl. A61K 31/765; C08L 85/02; C08L 77/00; C08L 67/00; H01M 10/02; A01P 3/00; A01P 13/00; C10L 1/04; A01P 21/00; A01P 7/04; H01G 9/004; C11D 3/36; C08L 1/02; A01N 57/20

(52) U.S. Cl. 424/78.08; 525/538; 525/420; 525/418; 525/419; 524/35; 521/134; 504/127; 510/469; 429/203; 252/62.2; 208/17

(57) ABSTRACT

The invention relates to mixtures of compounds of the formulae (I), (III), (IV), and/or (V),

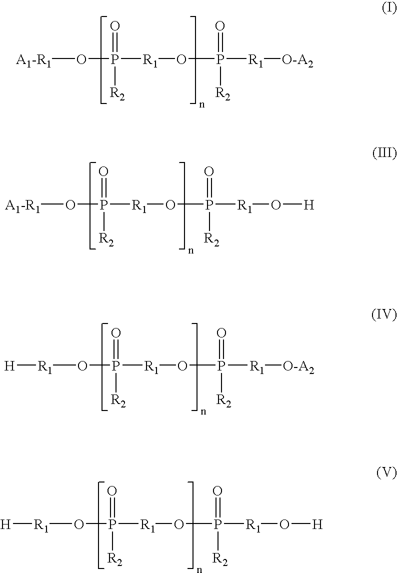

in which n is an average chain length of from 0 to 10,
A₁ and A₂ are identical or different and, independently of one another, are C(═O)NHR₅, C(═O)R₅, CH₃, CH₂CH₃, CH₂CH₂CH₃, CH(CH₃)₂, CH₂(CH₂)₂CH₃, CH₂CH(CH₃)₂, C(CH₃)₃, C₆H₅, and/or CH₂—C₆H₅,
R₁ is a moiety of the formula (II),

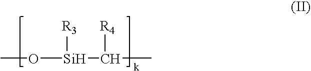

in which k is an average chain length of from 0 to 5;
R₂ is CH₃, CH₂CH₃, CH₂CH₂CH₃, CH₂(CH₃)₂, CH₂(CH₂)₂CH₃, CH₂CH(CH₃)₂, C(CH₃)₃, C₆H₅, or CH₂—C₆H₅; OCH₃, OCH₂CH₃, O(CH₂)₂CH₃, OCH(CH₃)₂, O(CH₂)₃CH₃, OCH₂CH(CH₃)₂, OC(CH₃)₃, O—C₆H₅, or O—CH₂—C₆H₄,
R₃, R₄, R₅ are identical or different and, independently of one another, are CH₃, CH₂CH₃, CH₂CH₂CH₃, CH(CH₃)₂, CH₂(CH₂)₂CH₃, CH₂Ch(CH₃)₂, C(CH₃)₃, C₆H₅, and/or CH₂—C₆H₅ and where at least 2 compounds of the formulae (I), (III), (IV), or (V) are simultaneously present in the mixture; to a process for their preparation, and to their use as flame retardants.

The present invention is described in the German priority application No. 102007056532.3, filed Nov. 23, 2007, which is hereby incorporated by reference as is fully disclosed herein.

The invention relates to mixtures of phosphorus-containing compounds, to a process for their preparation, and to their use as flame retardants.

Phosphorus-containing compounds have many uses as flame retardants in various plastics, for example in polyurethanes. Polyurethane plastics can be processed to give foams, foils, and casting resins, and are used in many sectors, e.g. in furniture, mattresses, transport, construction, and technical insulation. In order to comply with stringent flame retardancy requirements such as those demanded for materials used inter alia for the fitting-out of automobile interiors, of rail vehicle interiors, and of aircraft interiors, and also buildings insulation, polyurethane plastics have to be provided with highly efficient flame retardants. There are many different flame retardants known and commercially available for this purpose. However, the use of these flame retardants is often hindered by considerable technical problems with their use, or toxicological concerns.

For example, when solid flame retardants are used, examples being melamine, ammonium polyphosphate, and ammonium sulfate, problems with metering technology arise and often necessitate modifications to the foaming systems, i.e. complicated re-engineering and adaptation of these systems. A feature of many of the liquid flame retardants used, examples being tris(2-chloroethyl)phosphate, tris(2-chloroisopropyl)phosphate, and tetrakis(2-chloroethyl)ethylenediphosphate, is a marked tendency toward migration, which substantially restricts usefulness in open-cell flexible polyurethane foam systems for the fitting-out of automobile interiors, because of the requirements relating to condensable emissions (fogging).

Fogging is condensation of vaporized volatile constituents from interior fittings of the motor vehicle on panes of glass, in particular the windshield. DIN 75201 permits quantitative assessment of this phenomenon.

Halogen-free flame retardant systems are moreover preferred for reasons of environmental toxicology, and also because of their better ancillary properties in terms of smoke density and smoke toxicity in the event of a fire. Halogen-free flame retardants can also be of particular interest for technical reasons associated with their use; by way of example, marked corrosion is observed on the plant components used for the flame lamination of polyurethane foams when halogenated flame retardants are used. This can be attributed to the hydrogen halide emissions arising during flame lamination of halogen-containing polyurethane foams.

Flame lamination is the name given to a process for the bonding of textiles and foams where a flame is used for incipient melting of one side of a foam foil and this is then immediately laminated to a textile web.

In the light of the trend toward consideration of gaseous emissions (Volatile Organic Compounds=VOC), there are moreover increasingly stringent requirements relating to the migration resistance of flame retardants.

High migration resistance is provided by way of example by hydroxyalkyl phosphonates (DE-A-19 927 548) and oligomeric phosphoric esters bearing hydroxy groups (DE-A-4 342 973). These phosphoric esters and hydroxyalkyl phosphonates exhibit only low fogging values, but high susceptibility to hydrolysis, and a tendency toward closed-cell character in the polyurethane foam, resulting in impairment of the mechanical properties of the foam. A further disadvantage consists in the high hydroxy number, giving the flame retardant high reactivity toward the isocyanate component and thus making control of foam production more difficult.

It is an object of the present invention to provide phosphorus-containing compounds which when used as flame retardants eliminate the abovementioned disadvantages.

Another object of the present invention is to provide a flame retardant which does not have the abovementioned disadvantages. A particular intention is that when the flame retardant of the invention is used for flexible polyurethane foams the resultant fogging values are low and the foam has appropriate open-cell character. The flame retardant itself is intended to have low susceptibility to hydrolysis and a low hydroxy number, thus making it easier to meter the flame retardant and to control foam production.

The invention therefore provides mixtures of compounds of the formulae (I), (III), (IV), and/or (V),

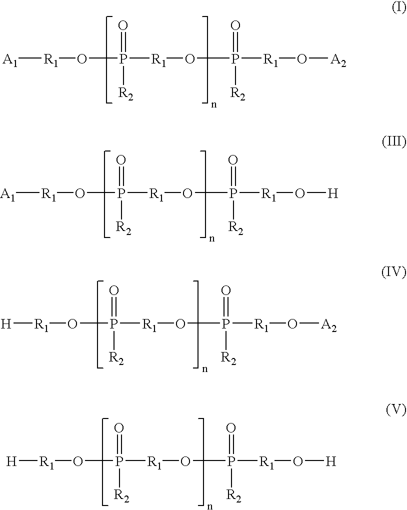

in which n is an average chain length of from 0 to 10,

A₁ and A₂ are identical or different and, independently of one another, are C(═O)NHR₅, C(═O)R₅, CH₃, CH₂CH₃, CH₂CH₂CH₃, CH(CH₃)₂, CH₂(CH₂)₂CH₃, CH₂CH(CH₃)₂, C(CH₃)₃, C₆H₅, and/or CH₂—C₆H₅,

R₁ is a moiety of the formula (II),

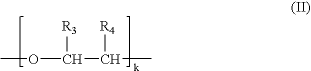

in which k is an average chain length of from 0 to 5;

R₂ is CH₃, CH₂CH₃, CH₂CH₂CH₃, CH₂(CH₃)₂, CH₂(CH₂)₂CH₃, CH₂CH(CH₃)₂, C(CH₃)₃, C₆H₅, or CH₂—C₆H₅; OCH₃, OCH₂CH₃, O(CH₂)₂CH₃, OCH(CH₃)₂, O(CH₂)₃CH₃, OCH₂CH(CH₃)₂, OC(CH₃)₃, O—C₆H₅, or O—CH₂—C₆H₅,

R₃, R₄, R₅ are identical or different and, independently of one another, are CH₃, CH₂CH₃, CH₂CH₂CH₃, CH(CH₃)₂, CH₂(CH₂)₂CH₃, CH₂CH(CH₃)₂, C(CH₃)₃, C₆H₅, and/or CH₂—C₆H₅ and where at least 2 compounds of the formulae (I), (III), (IV), or (V) are simultaneously present in the mixture.

It is preferable that the mixtures comprise

from 0.1 to 99.7% by weight of compounds of the formula (I),

from 0.1 to 99.7% by weight of compounds of the formula (II), and

from 0.1 to 99.7% by weight of compounds of the formula (IV).

It is also preferable that the mixtures comprise

from 0.1 to 99.6% by weight of compounds of the formula (I),

from 0.1 to 99.6% by weight of compounds of the formula (II),

from 0.1 to 99.6% by weight of compounds of the formula (IV), and

from 0.1 to 99.6% by weight of compounds of the formula (V).

It is preferable that

R₂ is CH₃, CH₂CH₃, CH₂CH₂CH₃, CH(CH₃)₂, C₆H₅, OCH₃, OCH₂CH₃, O(CH₂)₂CH₃, or O—C₆H₅,

R₃, R₄, and R₅ are identical or different and, independently of one another, are CH₃, CH₂CH₃, and/or CH₂CH₂CH₃, and

A₁ and A₂ are identical or different and, independently of one another, are C(═O)NHR₅, C(═O)R₅, CH₃, CH₂CH₃, (CH₂)₂CH₃, CH(CH₃)₂, CH₂(CH₂)₂CH₃, CH₂CH(CH₃)₂, C(CH₃)₃, C₆H₅, and/or CH₂—C₆H₅,

k is an average chain length of from 1 to 3, and

n is an average chain length of from 0 to 5.

The invention also provides a process for the preparation of the abovementioned mixtures as claimed in one or more of claims 1 to 3, which comprises reacting a polymeric phosphoric or phosphonic ester of the formula (V) with a capping reagent in the presence of a basic catalyst, to give the mixture.

It is preferable that the capping reagent involves monoalkyl isocyanates, monoaryl isocyanates, carboxylic anhydrides, carbonyl chlorides, alkyl halides, aralkyl halides, and/or diazomethane.

It is preferable that the basic catalyst involves tertiary amines and/or organic zinc compounds.

It is preferable that the basic catalyst involves pyridine, triethylamine, tetramethylbutanediamine (TMBDA), 1,4-diaza[2.2.2]bicyclooctane (DABCO), tetramethylguanidine, 1,5-diazabicyclo[4.3.0]non-5-ene (DBN), 1,8-diazabicyclo[5.4.0]undec-7-ene (DBU), dibutyltin dilaurate, stannous octoate, and/or stannous ricinoleate.

It is particularly preferable that the basic catalyst involves stannous octoate and/or pyridine.

The invention also provides the use of the abovementioned mixtures as claimed in one or more of claims 1 to 3 as flame retardants, or in a mixture with other flame retardants, or alone or in a mixture with other flame retardants for the preparation of further flame retardants or of precursors thereof.

It is preferable that the abovementioned mixtures as claimed in one or more of claims 1 to 3 are used as flame retardants for polyurethane plastics which are based on polyether systems or on polyester systems.

The invention in particular provides the use of the abovementioned mixtures as claimed in one or more of claims 1 to 3 as flame retardants for the production of flame-retardant polyurethane plastics which are produced in the form of elastomers by casting, in the form of rigid or flexible foams by a continuous or batchwise production method, or in the form of foamed or solid molded items, or in the form of foil.

It is also preferable that the abovementioned mixtures as claimed in one or more of claims 1 to 3 are used as flame retardant boosters, as flame retardants for clearcoat lacquers and intumescent coatings, or flame retardants for wood and other cellulose-containing products, or as reactive and/or non-reactive flame retardant for polymers, and/or for providing flame retardancy to polyester and to unblended or blended cellulose textiles, by impregnation.

However, the invention also provides the use of the abovementioned mixtures as claimed in one or more of claims 1 to 3 as binders for foundry materials and molding sands;

as crosslinking agents or accelerator in the hardening of epoxy resins, of polyurethanes, or of unsaturated polyester resins;

as polymer stabilizers, e.g. as light stabilizer, free-radical scavengers, and/or heat stabilizers for cotton fabrics, polymer fibers, plastics;

as crop protection agent, e.g. as plant growth regulator, or as herbicide, pesticide, or fungicide;

as therapeutic agent or additive in therapeutic agents for humans and animals, e.g. as enzyme modulator, for stimulation of tissue growth;

as sequestering agent, in petroleum production, and in metal-treatment agents;

as petroleum additive, e.g. as antioxidant, and for increasing octane number;

or as corrosion-prevention agent;

in laundry-detergent and cleaning-product applications, e.g. as decolorizer;

in electronics applications, e.g. in polyelectrolytes for capacitors, batteries, and accumulators, or else as free-radical scavengers in photosensitive layers;

as aldehyde scavengers;

or formaldehyde scavengers in adhesive compositions and in moldings, e.g. in

construction applications, in the automobile industry, in shipbuilding, in the aerospace industry, and for electrical engineering.

The object mentioned in the introduction is also solved via a flame retardant comprising a mixture of compounds of the formulae (I), (III), (IV), and/or (V), which comprises

from 0.1-10% by weight of compounds of the formula (V),

from 0-30% by weight of compounds of the formula (III) and (IV), and

from 70-99.9% by weight of compounds of the formula (I).

Likewise suitable is a flame retardant comprising a mixture of compounds of the formulae (I), (III), (IV), and/or (V), which comprises

from 0.1-20% by weight of compounds of the formula (V),

from 50-99.8% by weight of compounds of the formula (III) and (IV), and

from 0.1-20% by weight of compounds of the formula (I).

In another embodiment, the flame retardant comprises a mixture of compounds of the formulae (I), (III), (IV), and/or (V), in the following amounts:

from 0.1-10% by weight of compounds of the formula (I),

from 25-60% by weight of compounds of the formulae (III) and (IV), and

from 30-74.9% by weight of compounds of the formula (V).

In a further embodiment, the flame retardant comprises a mixture of compounds of the formulae (I), (III), (IV), and/or (V), in the following amounts:

from 0.1-5% by weight of compounds of the formula (I),

from 10-40% by weight of compounds of the formulae (III) and (IV), and

from 50-89.9% by weight of compounds of the formula (V).

It is preferable that R₂ in the formulae (I), (III), (IV), and/or (V) here is CH₃, CH₂CH₃, CH₂CH₂CH₃, CH(CH₃)₂, C₆H₅, OCH₃, OCH₂CH₃, O(CH₂)₂CH₃, or O—C₆H₅, R₃, R₄, and R₅ are identical or different and, independently of one another, are CH₃, CH₂CH₃, and/or CH₂CH₂CH₃, and

A₁ and A₂ are identical or different and, independently of one another, are C(═O)NHR₅, C(═O)R₅, CH₃, CH₂CH₃, (CH₂)₂CH₃, CH(CH₃)₂, CH₂(CH₂)₂CH₃, CH₂CH(CH₃)₂, C(CH₃)₃, C₆H₅, and/or CH₂—C₆H₅,

k is an average chain length of from 1 to 3, and

n is an average chain length of from 0 to 5.

The amount of compounds of the formulae (I), (III), (IV), and/or (V) present in the mixtures of the invention can in principle be regulated by way of the amounts introduced or, respectively, the usage ratio, of capping reagent to compound (V)—which is one of the starting materials. Accordingly, the quantitative proportions of compounds of the formulae (I), (III), (IV), and/or (V) in the mixture of the invention can vary within wide limits, corresponding to claims 2 and 3 and as given for the preferred mixtures on the preceding pages.

For example, if the usage ratio of phosphorus component to capping reagent is 1:2, the product is mixtures which comprise from 0.1 to 10% of polymeric phosphoric or phosphonic esters of the formula (V), a total of from 0 to 30% by weight of the phosphorus components of the formulae (III) and (IV), and from 70 to 99.9% by weight of the phosphorus components of the formula (I). The ratio of the phosphorus components present of the formulae (III) and (IV) here is preferably from 1.5:1 to 1:1.5.

If the usage ratio of phosphorus component to capping reagent is 1:1, the product is mixtures which comprise from 0.1 to 20% of polymeric phosphoric or phosphonic esters of the formula (V), a total of from 50 to 99.9% by weight of the phosphorus components of the formulae (III) and (IV), and from 0.1 to 20% by weight of the phosphorus components of the formula (I). The ratio of the phosphorus components present of the formulae (III) and (IV) is preferably from 1.5:1 to 1:1.5.

If the usage ratio of phosphorus component to capping reagent is 2:1, the product is mixtures which comprise from 30 to 75% by weight of polymeric phosphoric or phosphonic esters of the formula (V), a total of from 25 to 60% by weight of the phosphorus components of the formulae (III) and (IV), and from 0.1 to 10% by weight of the phosphorus components of the formula (I). The ratio of the phosphorus components present of the formulae (III) and (IV) is preferably from 1.5:1 to 1:1.5.

If the usage ratio of phosphorus component to capping reagent is 4:1, the product is mixtures which comprise from 50 to 90% of polymeric phosphoric or phosphonic esters of the formula (V), a total of from 10 to 40% of the phosphorus components of the formulae (III) and (IV), and from 0.1 to 5% of the phosphorus components of the formula (I). The ratio of the phosphorus components present of the formulae (III) and (IV) is preferably from 1.5:1 to 1:1.5.

It is preferable that the acid number of the flame retardant of the invention is from 0.05 to 5 mg KOH/g, particularly from 0.1 to 2 mg KOH/g. It is preferable that the viscosity of the flame retardant of the invention is from 0.01 to 50 Pa·s at 25° C., particularly from 5 to 15 Pa˜s at 25° C.

It is preferable that the phosphorus content of the flame retardant of the invention is from 1 to 25%, particularly from 5 to 20%.

It is preferable that the hydroxy number of the flame retardant of the invention is from 0 to 300 mg KOH/g, particularly from 5 to 200 mg KOH/g.

It is preferable that the water content of the flame retardant of the invention is from 0.001 to 2 mg/g, particularly from 0.01 to 0.9 mg/g.

The flame retardant of the invention is in particular characterized by being a clear to slightly cloudy liquid with a Hazen color number of from 1 to 1000, particularly preferably with a Hazen color number of from 1 to 400.

Surprisingly, it has been found that the flame retardant of the invention is unlike the corresponding uncapped polymeric phosphoric or phosphonic esters of the formula (V) in featuring relatively low susceptibility to hydrolysis and improved open-cell character in a PU foam in which it is used.

As starting material in the process of the invention it is preferable to use polymeric phosphoric or phosphonic esters of the formula (V), e.g. phosphoric ethylene glycol polyesters, phosphoric propylene glycol polyesters, alkylphosphonic ethylene glycol polyesters, alkylphosphonic propylene glycol polyesters, arylphosphonic ethylene glycol polyesters, and arylphosphonic propylene glycol polyesters.

As starting material, it is also preferable to use the polymeric phosphoric or phosphonic esters of the formula (V) such as methylphosphonic ethylene glycol polyesters, ethylphosphonic propylene glycol polyesters, propylphosphonic ethylene glycol polyesters, butylphosphonic ethylene glycol polyesters, methylphosphonic propylene glycol polyesters, ethylphosphonic propylene glycol polyesters, propylphosphonic propylene glycol polyesters, and butylphosphonic propylene glycol polyesters.

As starting materials (polymeric phosphoric or phosphonic esters of the formula (V)), it is particularly preferable to use the commercially available products Exolit® OP 550, Exolit® OP 560, and Exolit® OP 570.

As monoalkyl isocyanate, it is preferable to use methyl isocyanate, ethyl isocyanate, and cyclohexyl isocyanate. As monoaryl isocyanate, it is preferable to use benzyl isocyanate and phenyl isocyanate.

As carboxylic anhydride, it is preferable to use acetic anhydride and benzoic anhydride. As carbonyl chloride, it is preferable to use acetyl chloride and benzoyl chloride.

As alkyl halides, it is preferable to use methyl chloride, ethyl chloride, n-propyl chloride, isopropyl chloride, n-butyl chloride, isobutyl chloride, and tert-butyl chloride. As aralkyl halide, it is preferable to use benzyl chloride.

It is preferable that the polymeric phosphoric or phosphonic esters of the formula (V) are reacted in a molar ratio of from 1:2 to 5:1 with the capping reagent, particularly preferably in a molar ratio of from 1:1 to 1:2.

The molecular weight of the polymeric phosphoric or phosphonic esters here is calculated from their hydroxy number.

It is preferable to use from 0.001 to 50% of the basic catalyst, based on the polymeric phosphoric or phosphonic esters of the formula (V), particularly from 0.05 to 35%.

The process can be carried out by using the polymeric phosphoric or phosphonic esters of the formula (V) and the basic catalyst as initial charge, and adding the capping reagent dropwise.

The process can also be carried out by using the capping reagent and the basic catalyst as initial charge, and adding the polymeric phosphoric or phosphonic esters of the formula (V) dropwise.

As basic catalysts, it is preferable to use tertiary amines and salts of weak bases. Particularly preferred tertiary amines are pyridine, triethylamine, tetramethylbutanediamine (TMBDA), 1,4-diaza[2.2.2]bicyclooctane (DABCO), tetramethylguanidine, 1,5-diazabicyclo[4.3.0]non-5-ene (DBN), 1,8-diazabicyclo[5.4.0]undec-7-ene (DBU), dibutyltin dilaurate, stannous octoate, and stannous ricinoleate. Particular preference is given to pyridine and stannous octoate. As stannous octoate, particular preference is given to the commercially available products Desmorapide SO (Rhein Chemie) and Kosmos® 29 (Degussa).

It is preferable that the feed time is from 0.1 to 10 h, particularly from 1 to 5 h.

It is preferable that the after-reaction time is from 0.1 to 48 h, particularly from 1 to 10 h.

It is preferable that the reaction between polymeric phosphoric or phosphonic esters of the formula (V) and capping reagent is carried out at from 0 to 200° C., particularly at from 10 to 150° C. The invention also provides the use of the mixtures of the invention or, respectively, of the flame retardant of the invention, as intermediate for the preparation of flame retardants.

The invention provides flame retardants which comprise from 0.01 to 99.99% of the flame retardant of the invention and from 0.01 to 99.99% of other flame retardants.

Polyurethane prepolymer (molding compositions) The invention provides semiprepolymers and/or prepolymers which comprise from 0.01 to 99.99% of the flame retardant of the invention.

The invention also provides a process for the preparation of polyurethane prepolymers or, respectively, semiprepolymers, which comprises mixing from 0.01 to 50% of the flame retardant of the invention and from 0.01 to 99.99% of polyol with one another.

The invention provides polyurethane plastics which comprise from 0.01 to 99.99% of the flame retardant of the invention.

It is preferable that these involved foams have urethane groups and/or isocyanurate groups and/or allophanate groups and/or uretdione groups and/or urea groups and/or carbodiimide groups.

Polyisocyanate Component

Polyurethane plastics mainly involve foams having urethane groups and/or isocyanurate groups and/or allophanate groups and/or uretdione groups and/or urea groups and/or carbodiimide groups. The use according to the invention preferably takes place during the production of polyurethane plastics.

Polyol Component

Other starting components are compounds having at least two hydrogen atoms reactive toward isocyanates and having a molecular weight of from 400 to 10 000 (“polyol component”). These are not only compounds having amino groups but also compounds having thio groups or carboxy groups, and preferably compounds having hydroxy groups, in particular compounds having from 2 to 8 hydroxy groups, specifically those of molecular weight from 1000 to 6000, preferably from 200 to 6000, and generally, as is known per se for the production of homogeneous and of cellular polyurethanes, polyester amides and polycarbonates, and also polyesters and polyethers generally having from 2 to 8, but preferably from 2 to 6, hydroxy groups. The polyesters and polyethers having at least two hydroxy groups are preferred in the invention.

Further starting components are compounds having at least two hydrogen atoms reactive toward isocyanates and having a molecular weight of from 32 to 399. In this instance too, these are compounds having hydroxy groups and/or amino groups and/or thio groups and/or carboxy groups, preferably compounds having hydroxy groups and/or amino groups, these compounds serving as chain extenders or crosslinking agents. These compounds generally have from 2 to 8, preferably from 2 to 4, hydrogen atoms reactive toward isocyanates.

Suitable blowing agents are water and/or volatile organic substances, e.g. n-pentane, isopentane, cyclopentane, halogenated alkanes, such as trichloromethane and methylene chloride, or chlorofluoroalkanes, CO₂, etc.

If appropriate, concomitant use is made of auxiliaries and additives, such as catalysts of the type known per se, surfactant additives, such as emulsifiers and foam stabilizers, reaction retarders, e.g. acidic substances, such as hydrochloric acid or organic acyl halides, and also cell regulators of the type known per se, e.g. paraffins or fatty alcohols, and dimethylpolysiloxanes, and also pigments or dyes, and further flame retardants of the type known per se, and also stabilizers with respect to the effects of aging and of weathering, and plasticizers, and substances having fungistatic or bacteriostatic action, and also fillers, such as barium sulfate, kieselghur, carbon black, or whiting.

The invention also provides the use of the mixtures of the invention or, respectively, of the flame retardant of the invention, as flame retardants for the production of flame-retardant polyurethane plastics which are produced in the form of elastomers by casting, in the form of rigid or flexible foams by a continuous or batchwise production method, or in the form of foamed or solid molded items, or in the form of foil.

The invention also provides the use of the mixtures of the invention or, respectively, of the flame retardant of the invention, as flame retardants for polyurethane plastics in the following applications: construction industry, refrigerator industry, technical insulation, insulating sheets, upholstery, textile inserts, mattresses, automobile seats, arm rests, and construction elements, and also seat coverings and dashboard coverings.

It is particularly preferable that the mixtures of the invention or, respectively, the flame retardant of the invention are used as flame retardants for polyurethane plastics which are based on polyether systems or on polyester systems.

The invention also provides a process for the production of flame-retardant flexible polyurethane foams characterized by low contributions to fogging and by appropriate open-cell character. The process for the production of said flexible polyurethane foams comprises reacting organic polyisocyanates with compounds having at least two hydrogen atoms reactive toward isocyanates, with conventional blowing agents, stabilizers, activators, and/or further conventional auxiliaries and additives, in the presence of the flame retardant of the invention, by the single-stage process, prepolymer process, or semiprepolymer process.

It is preferable that the mixtures of the invention or, respectively, the flame retardant of the invention are used as flame retardants for flexible polyurethane foams which are produced by a slab foaming process.

It is particularly preferable that the mixtures of the invention or, respectively, the flame retardant of the invention are used as flame retardants for flexible polyurethane foams which are then characterized by low contributions to fogging and by appropriate open-cell character.

### EXAMPLES

### Example 1

16 g (0.134 mol) of phenyl isocyanate were used as initial charge with 0.03 g of Desmorapid® SO (0.03%, based on Exolit OP 550), in a flask with dropping funnel, thermometer, and reflux condenser. 100 g of Exolit® OP 550 (hydroxy number: 131 mg KOH/g) were added dropwise, with stirring, at a temperature of from 70 to 80° C., and stirring was continued for some time. The resultant product of the invention is a clear, colorless liquid which features an acid number of 1.6 mg KOH/g, a hydroxy number of 66 mg KOH/g, and a viscosity of 8700 mPa·s at 25° C.

### Example 2

32 g (0.268 mol) of phenyl isocyanate were used as initial charge with 0.01 g of Desmorapids SO (0.01%, based on Exolit® OP 550), in a flask with dropping funnel, thermometer, and reflux condenser. 100 g of Exolit® OP 550 (hydroxy number: 145 mg KOH/g) were added dropwise, with stirring, at a temperature of from 80 to 85° C., and stirring was continued for some time. The resultant product of the invention is a clear, slightly yellowish liquid which features an acid number of 0.9 mg KOH/g, a hydroxy number of 13 mg KOH/g, and a viscosity of 9800 mPa·s at 25° C.

### Example 3

100 g of Exolit® OP 550 (hydroxy number: 139 mg KOH/g) were used as initial charge with 0.06 g of Desmorapid® SO (0.06%, based on Exolit OP 550), in a flask with dropping funnel, thermometer, and reflux condenser. Within a period of 2 h, 16 g (0.134 mol) of phenyl isocyanate were added dropwise, with stirring, at a temperature of from 60 to 70° C., and stirring was continued for some time. The resultant product of the invention is a clear, colorless liquid which features an acid number of 1.8 mg KOH/g, a hydroxy number of 71 mg KOH/g, and a viscosity of 9600 mPa·s at 25° C.

### Example 4

691 g of Exolit® OP 570 (TP) (hydroxy number: 438 mg KOH/g) were used as initial charge with 0.21 g of Desmorapide SO (0.03%, based on Exolit® OP 570), in a flask with dropping funnel, thermometer, and reflux condenser. Within a period of 2.5 h, 321 g (2.7 mol) of phenyl isocyanate were added dropwise, with stirring, at a temperature of from 75 to 80° C., and stirring was continued for some time. The resultant product of the invention is a clear, colorless liquid which features an acid number of 0.8 mg KOH/g, a hydroxy number of 155 mg KOH/g, and a viscosity of 10.3 Pa˜s at 25° C.

### Example 5

230 g of Exolit® OP 560 (hydroxy number: 417 mg KOH/g) were used as initial charge with 0.12 g of Desmorapid® SO (0.05%, based on Exolit® OP 560), in a flask with dropping funnel, thermometer, and reflux condenser. Within a period of 3 h, 64 g (0.9 mol) of ethyl isocyanate were added dropwise, with stirring, at a temperature of from 40 to 50° C., and stirring was continued for some time. The resultant product of the invention is a clear, colorless liquid which features an acid number of 0.5 mg KOH/g, a hydroxy number of 130 mg KOH/g, and a viscosity of 5500 mPa·s at 25° C.

### Example 6

100 g of Exolit® OP 560 (hydroxy number: 429 mg KOH/g) were used as initial charge with 0.01 g of Desmorapid® SO (0.01%, based on Exolit® OP 560), in a flask with dropping funnel, thermometer, and reflux condenser. Within a period of 1 h, 52 g (0.4 mol) of benzyl isocyanate were added dropwise, with stirring, at a temperature of from 85 to 90° C., and stirring was continued for some time. The resultant product of the invention is a clear, colorless liquid which features an acid number of 0.8 mg KOH/g, a hydroxy number of 137 mg KOH/g, and a viscosity of 10.1 Pa·s at 25° C.

### Example 7

100 g of Exolit® OP 550 (hydroxy number: 139 mg KOH/g) were used as initial charge in a flask with dropping funnel, thermometer, and reflux condenser. Within a period of 10 min, 13.7 g (0.134 mol) of acetic anhydride were first added dropwise, with stirring, at a temperature of from 20 to 30° C. 0.106 g (1.3 mmol) of dry pyridine (1 mol %, based on acetic anhydride) was then added dropwise, and the mixture was heated for 3 h to from 100 to 105° C., with stirring. The resultant product of the invention is a clear, pale yellow liquid featuring an acid number of 0.6 mg KOH/g, a hydroxy number of 63 mg KOH/g, and a viscosity of 4900 mPa·s at 25° C.

### Example 8

54.5 g (0.4 mol) of benzoic anhydride were melted in a flask with dropping funnel, thermometer, and reflux condenser, and 100 g of Exolit® OP 560 (hydroxy number: 429 mg KOH/g) were admixed at 60° C. 1.58 g (0.02 mol) of dry pyridine (5 mol %, based on benzoic anhydride) were then added, and the mixture was heated at from 85 to 90° C. for 5 h, with stirring. The resultant product of the invention is a clear, colorless liquid featuring an acid number of 0.7 mg KOH/g, a hydroxy number of 141 mg KOH/g, and a viscosity of 8200 mPa·s at 25° C.

### Example 9

100 g of Exolit® OP 560 (hydroxy number: 429 mg KOH/g) were used as initial charge in a flask with dropping funnel, thermometer, and reflux condenser. Within a period of 10 min, 40.8 g (0.4 mol) of acetic anhydride were first added dropwise, with stirring, at a temperature of from 20 to 30° C. 0.63 g (8 mmol) of dry pyridine (2 mol %, based on acetic anhydride) was then added dropwise, and the mixture was heated for 4 h to from 90 to 95° C., with stirring. The resultant product of the invention is a clear, colorless liquid featuring an acid number of 0.9 mg KOH/g, a hydroxy number of 132 mg KOH/g, and a viscosity of 5000 mPa·s at 25° C.

### Example 10

100 g of Exolit® OP 560 (hydroxy number: 429 mg KOH/g) were used as initial charge in a flask with two dropping funnels, thermometer, and reflux condenser. 31.4 g (0.4 mol) of acetyl chloride and 31.6 g (0.4 mol) of pyridine were then added dropwise at from 10 to 15° C., and the mixture was stirred for 10 h at 25° C. The resultant salt was removed by filtration. The resultant product of the invention is a clear, colorless liquid featuring an acid number of 1.2 mg KOH/g, a hydroxy number of 137 mg KOH/g, and a viscosity of 5100 mPa·s at 25° C.

### Example 11

100 g of Exolit® OP 550 (hydroxy number: 139 mg KOH/g) were used as initial charge in a flask with two dropping funnels, thermometer, and reflux condenser. 18.8 g (0.134 mol) of benzoyl chloride and 10.6 g (0.134 mol) of pyridine were then added dropwise at from 15 to 20° C., and the mixture was stirred for 5 h at 30° C. The resultant salt was removed by filtration. The resultant product of the invention is a clear, pale yellow liquid featuring an acid number of 1.5 mg KOH/g, a hydroxy number of 76 mg KOH/g, and a viscosity of 7900 mPa·s at 25° C.

### Example 12

Hydrolysis resistance of the flame retardant of the invention against Exolit® OP 570

To compare the hydrolysis resistance of Exolit® OP 570 (uncapped phosphoric or phosphonic ester) with the product resulting from Example 4, the acid number of the two products was determined prior to and after addition of 1% of water. Table 1 shows the acid number profile of the two products.

TABLE 1
Acid number profile in Example 12

| | Acid number [mg KOH/g] | |
|---|---|---|
| | Flame retardant | |
| Time | from Example 4 | Exolit ® OP 570 (TP) |
| Starting material | 0.8 | 1.4 |
| without H₂O addition | | |
| With 1% of H₂O | 1.3 | 1.4 |
| (immediately after | | |
| addition) | | |
| After 1 day | 2.5 | 5.3 |
| After 2 days | 2.7 | 6.7 |
| After 3 days | 3.1 | 8.6 |

The acid number of the flame retardant of the invention from Example 4 rises markedly more slowly after water addition than that of the corresponding uncapped product Exolit® OP 570. The flame retardant of the invention from Example 4 is therefore also markedly less susceptible to hydrolysis than Exolit® OP 570.

### Example 13

Flexible Polyurethane Foam Formulations and Testing

The Exolit® OP 570 flame retardant described above, and also the flame retardant of the invention from Example 4, were incorporated into flexible polyurethane foams with NCO index of 105, of formulation as in Table 2. The NCO index is an index describing the percentage ratio of the amount of isocyanate used with respect to the stoichiometric amount, i.e. the calculated amount of isocyanate for reaction of one isocyanate-active group with each isocyanate group.

TABLE 2
Flexible polyurethane foam formulation A

| | Standard | | Standard | |
|---|---|---|---|---|
| | without FR* | | with FR | |
| Polyether polyol | 100 | parts | 100 | parts |
| (Arcol ® Polyol 1108, Bayer | | | | |
| MaterialScience) | | | | |
| Tin ricinoleate | 0.3 | part | 0.3 | part |
| (Kosmos ® EF, Goldschmidt) | | | | |
| 2-((2-(2-(Dimethylamino)ethoxy)- | 0.2 | part | 0.2 | part |
| ethyl)methylamino)ethanol | | | | |
| (Tegoamin ® ZE3, Degussa) | | | | |
| Polyether-modified polysiloxane | 0.7 | part | 0.7 | part |
| (Tegostab ® B 8232, Goldschmidt) | | | | |
| Water | 3.5 | parts | 3.5 | parts |
| Tolylene 2,4- and 2,6-diisocyanate | 47.8 | parts | 47.8 | parts |
| (Desmodur ® T80, Bayer | | | | |
| MaterialScience) | | | | |
| Flame retardant | 0 | part | 1-10 | parts |

*FR: flame retardant

TABLE 3
Properties of flexible polyurethane foams A with flame
retardant of the invention from Example 4: comparison

| | | Flame retardant | |
|---|---|---|---|
| | | from Example 4 | |
| | Standard | (Exolit ® OP 570, | |
| | without FR | capped) | Exolit ® OP 570 |
| FR content | 0 | 7 | 4 |
| (parts) | | | |
| Full rise time (s) | 112 | 115 | 114 |
| Density (kg/m³) | 30.5 | 29.8 | 31.0 |
| Air permeability | 15 | 16 | >250 |
| FMVSS 302 fire | B = | SE | SE |
| classification | 82 mm/min | | |

The flame retardancy of the flexible polyurethane foams was tested by means of the FMVSS 302 test (Federal Motor Vehicle Safety Standard). Terminology for assessment of fire performance:

SE Self-extinguishing

SE/NBR Self-extinguishing/no combustion rate

SE/B Self-extinguishing/with combustion rate

B Combustion rate

The flexible PU foam A with the flame retardant of the invention from Example 4 exhibits substantially improved air permeability when compared with the flexible PU foam from the uncapped product Exolit® OP 570. The open-cell character of the flexible PU foam with the flame retardant from Example 4 is indeed just as good as that of the standard without flame retardant (this being highly unusual and very surprising, because the use of the flame retardants known hitherto from the prior art otherwise led to a reduction in open-cell character). The fire classification achieved by the flexible polyurethane foam A with the flame retardant from Example 4, SE, is the same as that achieved by the flexible polyurethane foam A using Exolit® OP 570 (TP).

TABLE 4
Flexible polyurethane foam formulation B

| | Standard | | Standard | |
|---|---|---|---|---|
| | without FR* | | with FR | |
| Polyether polyol | 100 | parts | 100 | parts |
| (Arcol ® Polyol 1108, Bayer | | | | |
| MaterialScience) | | | | |
| Triethylenediamine in | 0.2 | part | 0.2 | part |
| dipropylene glycol | | | | |
| (Tegoamin ® 33, Degussa) | | | | |
| Bis(2-dimethylaminoethyl) | 0.1 | part | 0.1 | part |
| ether in dipropylene glycol | | | | |
| (Niax ® catalyst A-1, GE | | | | |
| Silicones) | | | | |
| Stannous octoate | 0.15 | part | 0.15 | part |
| (Desmorapid ® SO; Rhein | | | | |
| Chemie) | | | | |
| Polyether-modified | 1.3 | parts | 1.3 | parts |
| polysiloxane | | | | |
| (Tegostab ® B 8232, | | | | |
| Goldschmidt) | | | | |
| Water | 3.5 | parts | 3.5 | parts |
| Tolylene 2,4- and 2,6- | 23.9 | parts | 23.9 | parts |
| diisocyanate | | | | |
| (Desmodur ® T80, Bayer | | | | |
| MaterialScience) | | | | |
| Tolylene 2,4- and 2,6- | 23.9 | parts | 23.9 | parts |
| diisocyanate | | | | |
| (Desmodur ® T65, Bayer | | | | |
| MaterialScience) | | | | |
| Flame retardant | 0 | part | 1-10 | parts |

*FR: flame retardant

TABLE 5
Properties of flexible polyurethane foams B with flame
retardant from Example 4: comparison

| | | Flame retardant | |
|---|---|---|---|
| | | from Example 4 | |
| | Standard | (Exolit ® OP 570, | Exolit ® OP |
| | without FR | capped) | 570 |
| FR content (parts) | 0 | 7 | 4 |
| Full rise time (s) | 120 | 122 | 115 |
| Density (kg/m³) | 30.1 | 32.0 | 31.3 |
| Air permeability | 4 | 5 | >250 |
| FMVSS 302 fire | B = 114 mm/min | SE | SE |
| classification | | | |

The flexible PU foam B with the flame retardant of the invention from Example 4 exhibits substantially improved air permeability when compared with the flexible PU foam from the uncapped product Exolit® OP 570 (TP). The open-cell character of the flexible PU foam with the flame retardant from Example 4 is indeed just as good as that of the standard without flame retardant. The fire classification achieved by the flexible polyurethane foam B with the flame retardant from Example 4, SE, is the same as that achieved by the foam using Exolit® OP 570 (TP).

1. A mixture of compounds of the formulae (I), (III), (IV), or (V), wherein the mixture includes at least two compounds, wherein the at least two compounds are of different formulae 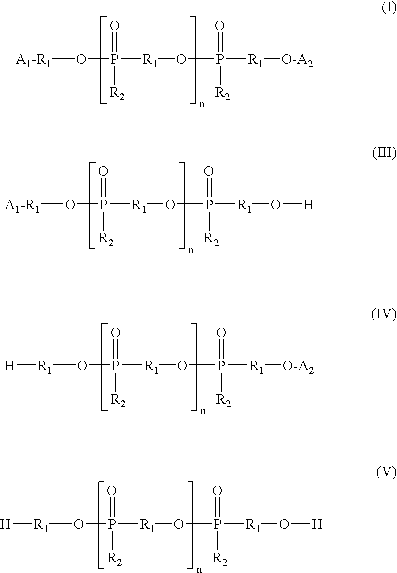
wherein n is an average chain length of from 0 to 10,
A₁ and A₂ are identical or different and, independently of one another, are C(═O)NHR₅, C(═O)R₅, CH₃, CH₂CH₃, CH₂CH₂CH₃, CH(CH₃)₂, CH₂(CH₂)₂CH₃, CH₂CH(CH₃)₂, C(CH₃)₃, C₆H₅, CH₂—C₆H₅,
R₁ is a moiety of the formula (II), 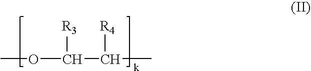
in which k is an average chain length of from 0 to 5;
R₂ is CH₃, CH₂CH₃, CH₂CH₂CH₃, CH₂(CH₃)₂, CH₂(CH₂)₂CH₃, CH₂CH(CH₃)₂, C(CH₃)₃, C₆H₅, or CH₂—C₆H₅; OCH₃, OCH₂CH₃, O(CH₂)₂CH₃, OCH(CH₃)₂, O(CH₂)₃CH₃, OCH₂CH(CH₃)₂, OC(CH₃)₃, O—C₆H₅, or O—CH₂—C₆H₅,
R₃, R₄, R₅ are identical or different and, independently of one another, are CH₃, CH₂CH₃, CH₂CH₂CH₃, CH(CH₃)₂, CH₂(CH₂)₂CH₃, CH₂CH(CH₃)₂, C(CH₃)₃, C₆H₅, or CH₂—C₆H₅.

2. The mixture of compounds as claimed in claim 1, comprising
from 0.1 to 99.7% by weight of compounds of the formula (I)
from 0.1 to 99.7% by weight of compounds of the formula (II)
from 0.1 to 99.7% by weight of compounds of the formula (IV).

3. The mixture of compounds as claimed in claim 1, comprising
from 0.1 to 99.6% by weight of compounds of the formula (I)
from 0.1 to 99.6% by weight of compounds of the formula (II)
from 0.1 to 99.6% by weight of compounds of the formula (IV)
from 0.1 to 99.6% by weight of compounds of the formula (V).

4. A process for the preparation of a mixture of compounds as claimed in claim 1, comprising the step of reacting a polymeric phosphoric or phosphonic ester of the formula (V) with at least one capping reagent in the presence of at least one basic catalyst, to form the mixture of compounds.

5. The process as claimed in claim 4, wherein the at least one capping reagent is selected from the group consisting of monoalkyl isocyanates, monoaryl isocyanates, carboxylic anhydrides, carbonyl chlorides, alkyl halides, aralkyl halides, and diazomethane.

6. The process as claimed in claim 4, wherein the at least one basic catalyst is selected from the group consisting of tertiary amines and organic zinc compounds.

7. The process as claimed in claim 4, wherein the at least one basic catalyst is pyridine, triethylamine, tetramethylbutanediamine (TMBDA), 1,4-diaza[2.2.2]bicyclooctane (DABCO), tetramethylguanidine, 1,5-diazabicyclo[4.3.0]non-5-ene (DBN), 1,8-diazabicyclo[5.4.0]undec-7-ene (DBU), dibutyltin dilaurate, stannous octoate, stannous ricinoleate or a mixture thereof.

8. The process as claimed in claim 1, wherein the at least one basic catalyst is stannous octoate, pyridine or a mixture thereof.

9. A flame retardant comnrising a mixture of compounds as claimed in claim 1.

10. A flame retardant for polyurethane plastics abased on polyether systems or on polyester systems, comprising a mixture of compounds as claimed in claim 1.

11. A flame retardant for the production of flame-retardant polyurethane plastics produced in the form of elastomers by casting, in the form of rigid or flexible foams by a continuous or batchwise production method, or in the form of foamed or solid molded items, or in the form of foil, comprising a mixture of compounds as claimed in claim 1.

12. A composition comprising a mixture of compounds as claimed in claim 1, wherein the composition is a flame retardant booster, flame retardant for clearcoat lacquers and intumescent coatings, flame retardant for wood and other cellulose-containing products, a reactive or non-reactive flame retardant for polymers.

13. A binder for foundry materials molding sands comprising a mixture of compounds as claimed in claim 1.

14. A flame retardant, comprising a mixture of compounds as claimed in claim 1, further comprising:
from 0.1-10% by weight of compounds of the formula (V),
from 0-30% by weight of compounds of the formula (III) and (IV), and
from 70-99.9% by weight of compounds of the formula (I).

15. A flame retardant, comprising a mixture of compounds as claimed is claim 1, further comprising:
from 0.1-20% by weight of compounds of the formula (V),
from 50-99.8% by weight of compounds of the formula (III) and (IV), and
from 0.1-20% by weight of compounds of the formula (I).

16. A flame retardant, comprising a mixture of compounds as claimed in claimed 1, further comprising:
from 0.1-10% by weight of compounds of the formula (I),
from 25-60% by weight of compounds of the formulae (III) and (IV), and
from 30-74.9% by weight of compounds of the formula (V).

17. A flame retardant, comprising a mixture of compounds as claimed in claim 1, further comprising:
from 0.1-5% by weight of compounds of the formula (I),
from 10-40% by weight of compounds of the formulae (III) and (IV), and
from 50-89.9% by weight of compounds of the formula (V).

18. The flame retardant as claimed in claim 14, wherein
R₂ is CH₃, CH₂CH₃, CH₂CH₂CH₃, CH(CH₃)₂, C₆H₅, OCH₃, OCH₂CH₃, O(CH₂)₂CH₃, or O—C₆H₅,
R₃, R₄, R₅ are identical or different and, independently of one another, are CH₃, CH₂CH₃, and/or or CH₂CH₂CH₃, and
A₁, A₂ are identical or different and, independently of one another, are C(═O)NHR₅, C(═O)R₅, CH₃, CH₂CH₃, (CH₂)₂CH₃, CH(CH₃)₂, CH₂(CH₂)₂CH₃, CH₂CH(CH₃)₂, C(CH₃)₃, C₆H₅, or CH₂—C₆H₅,
k is an average chain length of from 1 to 3, and
n is an average chain length of from 0 to 5.

19. A process for producing flame retardancy to a polyester or to unblended or blended cellulose textiles, comprising the step of impregnating a mixture of compounds as claimed in claim 1 to the polyester or the unblended or blended cellulose textile during production of the polyester or the unblended or blended cellulose textile.

20. A crosslinking agent or accelerator in the hardening of epoxy resins, polyurethanes or unsaturated polyester resins comprising a mixture of compounds as claimed in claim 1.

21. A polymer stabilizer, light stabilizer, free-radical scavenger or heat stabilizer for cotton fabrics, polymer fibers or plastics comprising a mixture of compounds as claimed in claim 1.

22. A crop protection agent, a plant growth regulator, or a herbicide, pesticide, or fungicide comprising a mixture of compounds as claimed in claim 1.

23. A therapeutic agent or additive in therapeutic agents for humans and animals, an enzyme modulator or tissue growth stimulizer comprising a mixture of compounds as claimed in claim 1.

24. A sequestering agent in petroleum production, and in metal-treatment agents comprising a mixture of compounds as claimed in claim 1.

25. A petroleum additive, antioxidant, an agent for increasing octane number or as corrosion-prevention agent comprising a mixture of compounds as claimed in claim 1.

26. A laundry-detergent or cleaning-product composition comprising a mixture of compounds as claimed in claim 1.

27 A polyelectrolyte for capacitors, batteries, and accumulators, or a free-radical scavenger in photosensitive layers comprising a mixture of compounds as claimed in claim 1.

28. An aldehyde scavenger comprising a mixture of compounds as claimed in claim 1.

29. A formaldehyde scavenger in adhesive compositions; in moldings for construction applications, the automobile industry, shipbuilding, the aerospace industry, and for electrical engineering.
